(19) United States
(12) Patent Application Publication
(10) Pub. No.: US 2026/0155255 A1
(43) Pub. Date: Jun. 4, 2026

(54) ATTENTION-BASED MULTIMODAL-FUSION FOR PATIENT SURVIVAL PREDICTION

(71) Applicant: Ventana Medical Systems, Inc., Tucson, AZ (US)

(72) Inventors: Ruining DENG, Nashville, TN (US); Nazim SHAIKH, Sunnyvale, CA (US); Gareth SHANNON, Walnut Creek, CA (US); Yao NIE, Sunnyvale, CA (US)

(21) Appl. No.: 19/458,443

(22) Filed: Jan. 23, 2026

Related U.S. Application Data
(63) Continuation of application No. PCT/US2024/040627, filed on Aug. 1, 2024.
(60) Provisional application No. 63/533,572, filed on Aug. 18, 2023.
(60) Provisional application No. 63/555,256, filed on Feb. 19, 2024.

(51) Int. Cl. G16H 50/20; G16H 30/20

(52) U.S. Cl. CPC G16H 50/20; G16H 30/20

(57) ABSTRACT

A method of predicting overall survivability of a patient by a prediction system based on machine learning includes receiving, by the prediction system, first modality data and second modality data corresponding to the patient; generating, by the prediction system, a first intermediate feature vector and a second intermediate feature vector based on the first and second modality data; determining, by the prediction system, a first attention score and a second attention score based on the first and second intermediate feature vectors; generating, by the prediction system, an aggregate feature vector based on the first and second intermediate feature vectors and the first and second attention scores; and generating, by the prediction system, a survivability prediction corresponding to the patient based on the aggregate feature vector.

### CROSS-REFERENCE TO RELATED APPLICATION(S)

This application is a by-pass continuation application of International Application No. PCT/US2024/040627, filed Aug. 1, 2024, which claims priority to, and the benefit of, U.S. Provisional Application No. 63/533,572 (“ATTENTION-BASED MULTIMODAL-FUSION FOR NON-SMALL CELL LUNG CANCER (NSCLC) PATIENT SURVIVAL PREDICTION”), filed on Aug. 18, 2023; and U.S. Provisional Application No. 63/555,256 (“ATTENTION-BASED MULTIMODAL-FUSION FOR PATIENT SURVIVAL PREDICTION”), filed on Feb. 19, 2024.

### FIELD

One or more aspects of some embodiments according to the present disclosure relate to a system and method for predicting patient outcomes.

### BACKGROUND

Cancers in their various forms have become one of the leading causes of death worldwide. In particular, lung cancer is one of the most prevalent malignancies and the cause of about 25% of all cancer-related deaths. About 84% of the lung cancers are non-small cell lung cancer (NSCLC), which is a group of lung cancers that behave similarly. Immunotherapy with checkpoint inhibitors, such as anti-PD1 and anti-PD-L1 drugs bring promising clinical outcomes for patients with locally advanced (ad) or metastatic (m) NSCLC. However, the biomarkers currently used in selecting patients who can benefit from the targeted or immunotherapy are inaccurate and have much potential for improvement.

Cancer prognosis and survival outcome prediction are crucial for therapeutic response prediction and the stratification of patients into different treatment groups. Integrating various data modalities into survival prediction models can enhance their predictive power, benefitting both clinical research and practice.

The above information disclosed in this Background section is only for enhancement of understanding of the background and therefore the information discussed in this Background section does not necessarily constitute prior art.

### SUMMARY

Aspects of embodiments of the present disclosure are directed to a multi-modal predictive system that utilizes a combination of histopathology and genomics data with an attention-based deep learning framework for predicting overall survival of patients (e.g., NSCLC patients). In some embodiments, attention-based deep learning framework utilizes a cross-modality attention-based multimodal fusion (CM-MMF) approach, which integrates image and RNA-sequence modalities to achieve superior patient survival predictions. Here, the attention scores derived from the fusion layer may highlight the significance of each modality during fusion for clinical diagnosis.

According to some embodiments of the present disclosure, in a method of predicting overall survivability of a patient by a prediction system based on machine learning, the method comprising: receiving, by the prediction system, first modality data and second modality data corresponding to the patient; generating, by the prediction system, a first intermediate feature vector and a second intermediate feature vector based on the first and second modality data; determining, by the prediction system, a first attention score and a second attention score based on the first and second intermediate feature vectors; generating, by the prediction system, an aggregate feature vector based on the first and second intermediate feature vectors and the first and second attention scores; and generating, by the prediction system, a survivability prediction corresponding to the patient based on the aggregate feature vector.

According to some embodiments, the first modality data comprises histology hematoxylin and eosin (H&E) image data, and the second modality data comprises genetic sequencing data.

According to some embodiments, the H&E image data comprises a digitized image of a tissue sample of the patient that is stained with hematoxylin and eosin dyes, and the genetic sequencing data comprises mRNA gene expressions extracted from a tumorous tissue of the patient.

According to some embodiments, the receiving the first modality data and the second modality data comprises: receiving, by a first model of the prediction system, the first modality data; and receiving, by a second model of the prediction system, the second modality data.

According to some embodiments, the generating the first intermediate feature vector and the second intermediate feature vector comprises: generating, by the first model, the first intermediate feature vector based on the first modality data; and generating, by the second model, the second intermediate feature vector based on the second modality data.

According to some embodiments, the first model comprises an attention-based multiple instance learning (AMIL) model, and the second model comprises a feedforward neural network (FNN) model.

According to some embodiments, the determining the first attention score and the second attention score comprises: combining the first and second intermediate feature vectors to generate a combined feature; processing the combined feature by one or more convolution layers and an activation layer to generate first and second attention values corresponding to the first and second intermediate feature vectors; and converting the first and second attention values to the first and second attention score by a softmax function layer.

According to some embodiments, the combining the first and second intermediate feature vectors comprises: stacking the first and second intermediate feature vectors into a 2-dimensional array that is the combined feature, and wherein the one or more convolution layers are configured to perform 2-dimensional convolution on the 2-dimensional array.

According to some embodiments, the activation layer comprises a hyperbolic tangent (Tanh) function layer or a rectified linear unit (ReLU) function layer, and wherein a summation of the first and second attention scores is equal to 1.

According to some embodiments, the method further includes normalizing values of the first and second intermediate feature vectors before the combining the first and second intermediate feature vectors.

According to some embodiments, the determining the first attention score and the second attention score comprises: processing the first intermediate feature vector by one or more first convolution layers and a first activation layer to generate a first attention value corresponding to the first intermediate feature vector; processing the second intermediate feature vector by one or more second convolution layers and a second activation layer to generate a second attention value corresponding to the second intermediate feature vector; and converting the first and second attention values to the first and second attention scores by a softmax function layer.

According to some embodiments, each of the first and second activation layers comprises a hyperbolic tangent (Tanh) function layer or a rectified linear unit (ReLU) function layer, and wherein a summation of the first and second attention scores is equal to 1.

According to some embodiments, the one or more first convolution layers comprise kernel values that are not the same as those of the one or more second convolution layers.

According to some embodiments, the generating the aggregate feature vector comprises: scaling the first intermediate feature vector based on the first attention score to generate a first scaled intermediate feature vector; scaling the second intermediate feature vector based on the second attention score to generate a second scaled intermediate feature vector; and aggregating the first and second scaled intermediate feature vectors to generate the aggregate feature vector.

According to some embodiments, the aggregating the first and second scaled intermediate feature vectors comprises: concatenating the first and second scaled intermediate feature vectors to generate the aggregate feature vector, the aggregate feature vector having a length equal to a sum of lengths of the first and second scaled intermediate feature vectors.

According to some embodiments, the generating the survivability prediction comprises: generating, by a classifier of the prediction system, the survivability prediction corresponding to the patient based on the aggregate feature vector, wherein the survivability prediction corresponds to an overall survivability of the patient.

According to some embodiments, the survivability prediction comprises a range of values from a plurality of sequential ranges of values.

According to some embodiments, the method further includes transmitting the survivability prediction to a display device for display to a user.

According to some embodiments, in a prediction system for predicting overall survivability of a patient based on machine learning, the prediction system comprising: a first model configured to receive first modality data corresponding to the patient and to generate a first intermediate feature vector based on the first modality data; a second model configured to receive second modality data corresponding to the patient and to generate a second intermediate feature vector based on the second modality data; and an attention-based multimodal fusion circuit configured to determine a first attention score and a second attention score based on the first and second intermediate feature vectors, and to generate an aggregate feature vector based on the first and second intermediate feature vectors and the first and second attention scores.

According to some embodiments, the prediction system further includes a classifier configured to receive the aggregate feature vector and to generate a survivability prediction corresponding to the patient based on the aggregate feature vector.

### BRIEF DESCRIPTION OF THE DRAWINGS

Non-limiting and non-exhaustive embodiments according to the present disclosure are described with reference to the following figures, wherein like reference numerals refer to like parts throughout the various views unless otherwise specified.

FIG. 1 is a block diagram illustrating a prediction system, according to some embodiments of the present disclosure.

FIG. 2A illustrates a block diagram of a first model of the prediction system, according to some embodiments of the present disclosure.

FIG. 2B illustrates a block diagram of a second model of the prediction system, according to some embodiments of the present disclosure.

FIG. 3A illustrates a block diagram of an attention-based multimodal fusion circuit adopting a non-shared attention-based multimodal fusion approach, according to some embodiments of the present disclosure.

FIG. 3B illustrates a block diagram of an attention-based multimodal fusion circuit adopting a shared attention-based multimodal fusion approach, according to some other embodiments of the present disclosure.

FIG. 4A illustrates a table comparing the performance of prediction system with different systems of the related art across various modalities with different types of loss supervision, according to some embodiments of the present disclosure.

FIG. 4B illustrates a table comparing the performance of prediction system using sharing and non-sharing kernel layers and different activation functions, according to some embodiments of the present disclosure.

FIG. 5 is a flow diagram illustrating a process of predicting overall survivability of a patient by the prediction system, according to some embodiments of the present disclosure.

### DETAILED DESCRIPTION

Hereinafter, aspects of some example embodiments will be described in more detail with reference to the accompanying drawings, in which like reference numbers refer to like elements throughout. The present invention, however, may be embodied in various different forms, and should not be construed as being limited to only the illustrated embodiments herein. Rather, these embodiments are provided as examples so that this disclosure will be thorough and complete, and will fully convey the aspects and features of the present invention to those skilled in the art. Accordingly, processes, elements, and techniques that are not necessary to those having ordinary skill in the art for a complete understanding of the aspects and features of the present invention may not be described. Unless otherwise noted, like reference numerals denote like elements throughout the attached drawings and the written description, and thus, descriptions thereof will not be repeated. In the drawings, the relative sizes of elements, layers, and regions may be exaggerated for clarity.

Aspects of some embodiments of the present disclosure enable predicting the survival outcomes of patients with non-small cell lung cancer (NSCLC) using a combination of histopathology and genomics with an attention-based deep learning approach. In particular, some embodiments utilize a cross-modality attention-based multimodal fusion (CM-MMF) approach, which integrates image and RNA-sequence modalities to achieve superior patient survival predictions. In some embodiments, an attention-based fusion circuit produces attention values that highlight the significance of each modality during fusion for clinical diagnosis. An attention-based fusion architecture may be capable of achieving relatively improved multimodal patient survival prediction, for example, achieving a C-index of about 0.6587, and demonstrating the functionality that infuses the complementary knowledge from different modalities.

Cancer prognosis and survival outcome prediction may enable therapeutic response prediction and the stratification of patients into different treatment groups. The integration of various data modalities into survival prediction models may enhance their predictive power, benefitting both clinical research and practice. Some embodiments enable predicting the survival outcomes of patients with NSCLC using a combination of histopathology and genomics with an attention-based deep learning approach.

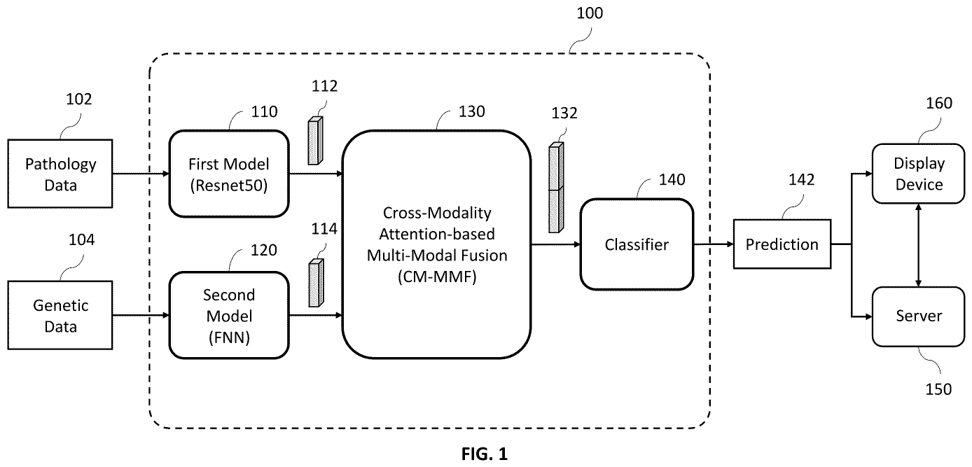

FIG. 1 is a block diagram illustrating a prediction system 100, according to some embodiments of the present disclosure.

According to some embodiments, the prediction system 100 is a multi-modality deep-learning framework that integrates various modality data and utilizes an attention mechanism to predict patient outcomes. In some embodiments, the prediction system 100 is configured to receive a first modality data 102 and a second modality data 104 that are associated with a patient and to generate a survivability prediction (e.g., a survivability score) 142 for the patient based on the modalities 102 and 104. The attention mechanism utilized by the prediction system 100 focuses the attention of the system to certain a modality, based on the different weights assigned to the different modalities and enhances prediction performance of the system 100.

The modalities that are utilized by the prediction system 200 may be of different types. For example, the first modality 102 may include histology hematoxylin and eosin (H&E) image data. The H&E data 102 may include one or more digitized images of a tissue sample (e.g., a tumorous tissue sample) of the patient that is stained with hematoxylin and eosin dyes. H&E dyes stain cell nuclei, extracellular matrix and cytoplasm, and other cell structures, with different colors thus allowing a pathologist and the prediction system 100 to differentiate between different cellular structure. Also, the overall patterns of coloration from the stain show the general layout and distribution of cells and provide a view of a tissue sample's structure. In some examples, the H&E image data 102 may include one or more image tiles that are extracted from (e.g., randomly selected and extracted from) a viable tumor region of a stained tissue sample.

The second modality 104 may include genetic sequencing data, such as DNA information and/or mRNA gene expressions of tumor mutation that are extracted from a tumorous tissue of the patient. Each tumor cell may have hundreds or thousands of tumor mutation genes. The second modality 104 may include some of the genetic mutations discovered in a tissue sample. In some example, only those expressions that are most relevant to patient survivability may be included in the second modality 104.

According to some embodiments, the prediction system 100 includes an attention-based fusion architecture to infuse first and second modalities (e.g., image and RNA-seq modalities) to achieve improved patient survival predictions. In some embodiments, the prediction system 100 includes a first model 110 that is configured to receive the first modality data 102 and to generate a first intermediate feature vector (e.g., a pathology/histology feature vector) 112, a second model 120 that is configured to receive the second modality data 104 and to generate a second intermediate feature vector (e.g., an omic/sequencing feature vector) 114. In some embodiments, the prediction system 100 further includes an attention-based multimodal fusion circuit (e.g., a cross-modality attention-based multi-modal fusion circuit (CM-MMF)) 130 that fuses the first and second intermediate feature vectors 112 and 114 to generate first and second weights (e.g., attention scores or scaling factors) for scaling the first and second intermediate feature vectors 112 and 114 based on their relative importance for the disease diagnosis, and generate an aggregate feature vector 132 based on the scaled first and second intermediate feature vectors. The prediction system 100 also includes a classifier 140 that generates a survivability prediction 142 of the patient based on the aggregate feature vector 132.

In some embodiments, the classifier 140 includes a neural network with a number of layers each of which may performs a convolutional operation, via the application of kernels/filters, on the aggregate feature vector 132. The neural network may, according to some examples, be a convolutional neural network (ConvNet/CNN), a recurrent neural network (RNN), a multilayer perceptron (MLP), or the like. However, embodiments of the present disclosure are not limited thereto, and the classifier 140 may, for example, include a single layer.

The survivability prediction 142 generated by the classifier 140 may correspond to an overall survivability of the patient. In some examples, the continuous timescale of overall patient survival time in days or months may be partitioned into a plurality of non-overlapping bins (e.g., four non-overlapping bins: bin 1 (1-200 days), bin 2 (201-400 days), bin 3 (401-600 days), etc.), and the output of the classifier 140 may be a particular bin from among the plurality of non-overlapping bins (e.g., a particular range of survivability days from among a plurality of sequential ranges of values. However, in some examples, the output of the classifier 140 may be a raw survivability score that indicates the number of days or months of patient survival.

Once the prediction system 100 generates a survivability prediction 142, the prediction may be transmitted to a server (e.g., a remote server or a cloud server) 150 for further processing and/or to a display device 160 for display to a user.

While the description above describes two modalities as examples of the input modalities 102 and 104 to the prediction system 100, embodiments of the present disclosure are not limited thereto, and any suitable type of modality may be employed by the prediction system 100 to generate the survivability prediction 142. For example, the prediction system 100 may utilize one or more derivates of the modalities 102 and 104.

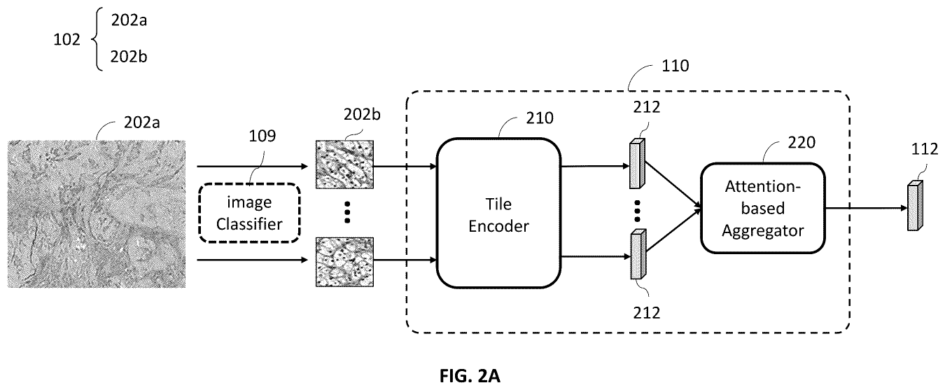

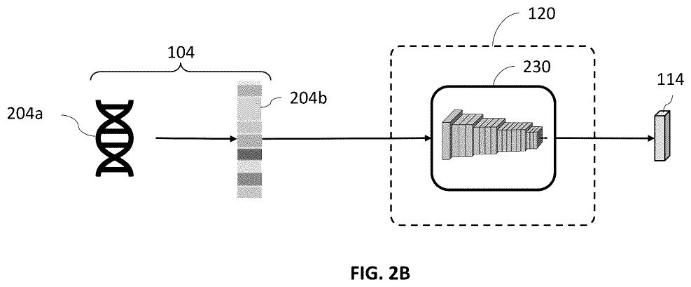

FIG. 2A illustrates a block diagram of the first model 110, according to some embodiments of the present disclosure.

In some examples, the pathology data 102 includes a digitized whole-slide image (WSI; e.g., a digitized image) 202a of a tissue sample slice from a patient, which contains tumor or cancer cells (e.g., lung cancer cells). The tissue sample slice may be stained with hematoxylin and eosin (H&E), which produce patterns of coloration that reveal the general layout and distribution of cells, differentiate different types of tissue, and provide a general overview of a tissue sample's structure. However, embodiments of the present disclosure are not limited thereto, and the tissue slice may be stained in any suitable manner so that the WSI 202a identifies the malignant (cancer) cells, non-squamous cells in NSCLC, and/or the like. The WSI 202a may be too large to process as a whole (e.g., it may be several gigapixels in size), and thus may be divided into smaller regions/sections, referred to herein as tiles (or patches) 202b, which are easier or more manageable to process. For example, an image classifier 109, such as a pretrained model with a U-Net architecture, may classify regions of the WSI 202a as tumor and stroma, and tiles 202b (e.g., 512×512 pixel tiles) may be extracted from the classified regions of the WSI 202a. The first model 110 may receive the tiles 202b or may receive the WSI 202a and subdivide it into the plurality of tiles 202b.

According to some embodiments, the first model 110 utilizes an attention-based multiple instance learning (AMIL) pipeline that extracts features 212 from each tile 204 of the WSI 202a and embeds the individual tile feature vectors 212 into the first intermediate feature vector 112. The first model 110 includes a tile encoder 210 and an attention-based aggregator 220.

The tile encoder 210 encodes each tile into a tile feature vector 212 (e.g., a 1024-channel feature vector). In some examples, the tile encoder 210 encodes each tile using a ResNet50-based image encoding architecture. However, embodiments of the present disclosure are not limited thereto, and any suitable image encoder, such as a convolutional neural network (CNN) may be utilized.

The attention-based aggregator 220 receives the tile feature vectors 212 and determines a tile weight for each tile based on its perceived relevance to patient-level prognostic prediction, allowing it to highlight important regions and to identify pivotal tiles when aggregating the tiles into a WSI feature representation. That is, the regions that receive high tile weights contribute more to the patient-level feature representation than those assigned tile weights. The tile weights are then used to create a slide level representation feature vector, that is the first intermediate feature vector 112. This may be achieved through an attention-pooling operation, which aggregates information from all regions in the patient's WSIs. In some examples, the first intermediate feature vector 112 may be a 1024 channel feature vector (e.g., a 1×1024 matrix) that is representative of the entire input WSI 202a. However, embodiments of the present disclosure are not limited thereto, and in some examples, the first model 110 may output a 128 channel feature vector (e.g., a 128×1 matrix) that is representative of the entire input WSI 202a.

FIG. 2B illustrates a block diagram of a second model 120, according to some embodiments of the present disclosure.

In some examples, the genetic data 104 includes bulk sequencing RNA data 204a from which preselected features 204b may be extracted. In some examples, the preselected features 204b may include 154 features from among thousands in the bulk sequencing RNA data 204a. However, embodiments of the present disclosure are not limited thereto, and any suitable number of pre-selected features 204b may be utilized. For example, the number of pre-selected features may differ for different locations of tissue (e.g., breast, lung, etc.) and type of cancer being analyzed. Further, the second modality 104 may include other molecular profile data such as mutation status, copy number variations, etc.

In some embodiments, the second model 120 includes a self-normalizing neural network (SNN) 230 or a feedforward neural network (FNN) to embed the extracted RNA-sequence information 204b into a feature vector for omic feature representation, that is, the second intermediate feature vector 114. In some examples, the second intermediate feature vector 114 may be a 128 channel feature vector. However, embodiments of the present disclosure are not limited thereto, and the feature vector 114 may include any suitable number of channels. Further, embodiments of the present disclosure are not limited to using an FNN network, and any suitable network, such as a self-normalizing network (SNN) may be utilized to process sequencing data.

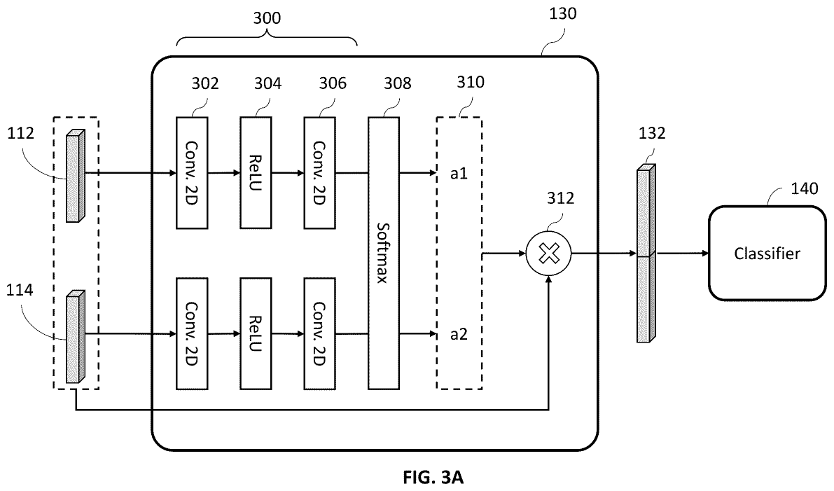

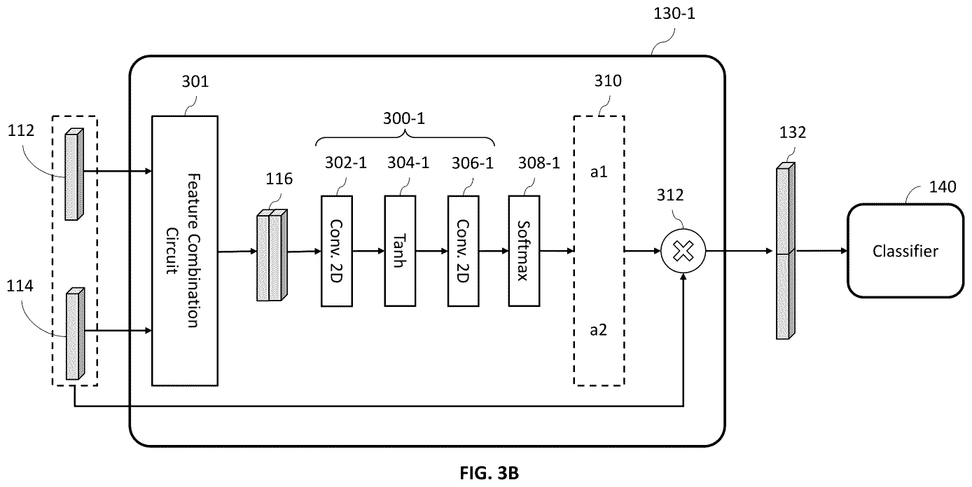

FIG. 3A illustrates a block diagram of the attention-based multimodal fusion circuit 130 adopting a non-shared attention-based multimodal fusion approach, according to some embodiments of the present disclosure.

In some embodiments, the attention-based multimodal fusion (AMMF) circuit 130 includes, for each modality data 102 and 104, a separate modality attention path 300 that considers the importance of that modality data for survival prediction. Each modality attention path 300 receives and processes a corresponding one of the first and second intermediate feature vectors 112 and 114. Each modality attention path 300 includes two convolutional layers 302 and 306 and an activation function layer 304 therebetween, which introduces a non-linearity into the model and improves how well the AMMF circuit 130 is trained (e.g., the activation function may produce a zero-centered output that supports the backpropagation process during training). The convolutional layers 302 and 306 with decreasing channel numbers compress features into more compact representations based on certain specific knowledge for future purposes. In some examples, the fully convolutional layers 302 and 306 may convolutional neural networks (CNNs) having a convolution kernel/filter size of 1×1 and the activation layer 304 may be a rectified linear unit (ReLU) function layer. However, embodiments of the present disclosure are not limited thereto, and the kernels of the convolutional layers 302 and 306 may have any suitable size, and any suitable non-linear activation function (such as a hyperbolic tangent (Tanh) function layer) may be used in place of the ReLU function layer.

In the embodiments of FIG. 3A, because the intermediate feature vectors 112 and 114 are processed by different modality attention path 300, the intermediate feature vectors 112 and 114 may not be homogenous, that is, may have different numerical ranges and/or may have different dimensions (e.g., different vector lengths). However, in other examples, the intermediate feature vectors 112 and 114 may be normalized to be homogenous, that is, to have the same numerical range (e.g., from 0 to 1) and/or may be dimensionally the same (e.g., have the same vector lengths).

As shown in FIG. 3A, the kernel weights of the convolutional layers 302 and 306 may not be shareable between the different modalities which leads to the AMMF circuit 130 learning complementary information. In other words, the AMMF of FIG. 3A may adopt a non-kernel-sharing approach. For example, the kernel values of the modality attention path 300 corresponding to the first modality data 102 may be different from those of the modality attention path 300 corresponding to the second modality data 104.

The attention-based multimodal fusion circuit 130 may also include a softmax layer 310, which assigns decimal probabilities to the outputs of the two separate modality attention paths 300. Accordingly, the softmax layer 310 outputs two attention scores a₁ and a₂ that add up to one and represent the relative weight/importance of the two input modalities 102 and 104 with regard to survivability prediction 142 of the prediction system 100.

In some embodiments, the cross-modality attention score (am) can be represented according to Equation (1):

$${a}_{m} = \frac{{exp W}_{m}^{T} ReLU ( {V}_{m} {f}_{m}^{T} )}{{\sum }_{m = 1}^{M} {exp W}_{m}^{T} ReLU ( {V}_{m} {f}_{m}^{T} )} \qquad Eq . ( 1 )$$

Where m is an integer index (modality), M is an integer representing the number of the input modalities 102/104 (e.g., 2), Wₘ∈R^{L×1} and Vₘ∈R^{L×N} are matrices of trainable parameters in the AMMF circuit 130 representing weights of two convolutional layers, L represents the size (e.g., length) of the unimodal embedding output fm (i.e., the size of the first/second intermediate feature vector 112/114), N is the number of output channels of the first layer of the AMMF circuit 130 (i.e., the size of the output of the first convolutional layer 302), T represents the matrix transpose operation, and ReLU(·) denotes the rectified linear unit activation function.

In some embodiments, the attention-based multimodal fusion circuit 130 also includes a feature aggregator (e.g., a feature aggregation circuit) 312 that applies the attention scores a₁ and a₂ to the corresponding ones of the first and second intermediate feature vectors 112 and 114 and combines the results to generate an aggregate feature vector 132. In some examples, the feature aggregator 312 may scale the first intermediate feature vector 112 based on the first attention score a₁ to generate a first scaled intermediate feature vector, may scale the second intermediate feature vector 114 based on the second attention score a₂ to generate a second scaled intermediate feature vector, and may aggregate (e.g., combine) the first and second scaled intermediate feature vectors to generate the aggregate feature vector 132. The scaling operation performed by the feature aggregator 312 may involve multiplying the attention score a₁/a₂ by the corresponding intermediate feature vector 112/114. In some examples, the aggregation process may involve concatenating the first and second scaled intermediate feature vector to generate the aggregate feature vector 132. However, embodiments of the present disclosure are not limited thereto.

According to some embodiments, the aggregation operation the feature aggregator 312 includes multiplying the cross-modality attention scores (am) with corresponding modality intermediate feature vectors 112/114 to yield a unified cross-modality representation Fₘ (i.e., the aggregate feature vector 132), which can be expressed by Equation (2):

$${F}_{m} = {\sum }_{□ = 1}^{□} {a}_{m} {f}_{m} \qquad Eq . ( 2 )$$

For final prediction, the classifier 140 (e.g., a one-layer classifier) is implemented to facilitate patient-wise survival prediction 142 using cross-modality embedding (Fₘ).

In the embodiments of FIG. 3A, the intermediate feature vectors 112 and 114 of the input modalities 102 and 104 are separately processed via two independent modality attention paths 300, which may have convolution layers with different kernels values for different modalities; however, embodiments of the present disclosure are not limited to this non-sharing attention-based multimodal fusion architecture. For example, the intermediate feature vectors corresponding to the input modalities may be processed by via a shared attention-based multimodal fusion architecture.

FIG. 3B illustrates a block diagram of the attention-based multimodal fusion circuit 130-1 adopting a shared attention-based multimodal fusion approach, according to some other embodiments of the present disclosure.

According to some embodiments, the attention-based multimodal fusion (AMMF) circuit 130-1 determines the relative importance of the two input intermediate feature vectors 112 and 114 via a shared/common attention-based multimodal fusion (AMMF) circuit 130-1. Here, because the intermediate feature vectors 112 and 114 (each of which may, e.g., be 128-channel feature vectors) are the outputs of two different models 110 and 120, they may not be homogenous, that is, may have different numerical ranges. In such examples, the AMMF circuit 130-1 includes a feature combination circuit 301 that normalizes (e.g., scales) the input intermediate feature vectors 112 and 114 to be homogenous, that is, to have the same numerical range (e.g., from 0 to 1), and combines the normalized feature into the combined feature 116 (e.g., a 128×2 feature vector) that is then processed by a single modality attention path 300-1. The normalization performed by the feature combination circuit 301 may be a scaling function or may be any suitable normalization technique that transforms the input features 112 and 144 into a common domain prior to being combined. While the feature combination circuit 301 is shown in FIG. 3B as being part of the AMMF circuit 130-1, embodiments of the present disclosure are not limited thereto, and the operation of the feature combination circuit 301 may be performed external to the AMMF circuit 130-1.

The combined feature 116 may be a concatenation of the normalized input features 112 and 114 into a one-dimensional vector with a length that is the sum of the lengths of the normalized feature vectors, or may be a stacking of the normalized input features 112 and 114 into a two-dimensional array in which each row/column includes a corresponding one of the two normalized input features (as shown in FIG. 3B). In the examples of FIG. 3B, the input intermediate feature vectors 112 and 114 may be dimensionally the same (e.g. may have the same vector length).

According to some embodiments, the common modality attention path 300-1 includes two convolutional layers 302-1 and 306-1, which may be two-dimensional convolutional layers, and an activation function layer 304-1 therebetween, which may be a hyperbolic tangent (Tanh) function layer. In some examples, the fully convolutional layers 302-1 and 306-1 may have a 1×1 convolution kernel/filter that is concurrently (e.g., simultaneously) applied to both dimensions of the combined feature 116. However, embodiments of the present disclosure are not limited thereto, and the kernels of the convolutional layers 302 and 306 may have any suitable size. Further, the activation layer 304-1 is not limited to a Tanh function layer, and may be any suitable non-linear activation function (such as a rectified linear unit (ReLU) function layer.

As shown in FIG. 3B, the AMMF circuit 130-1 of FIG. 3B may adopt a kernel-sharing approach in which the kernel weights of the convolutional layers 302-1 and 306-1 are shareable between the different modalities. That is, the intermediate feature vectors 112 and 114 from the different modalities 102 and 104 are convolved by the same kernel values. This serves to promote a holistic learning of the importance of modality-specific knowledge through cross-modality relationships.

The AMMF circuit 130-1 may also include a softmax layer 308-1, which may be the same or substantially the same as the softmax layer 308 of FIG. 3A and is configured to output attention scores (e.g., cross-modality attention scores/weights) a₁ and a₂ that add up to one and represent the relative weight/importance of the two input modalities 102 and 104 with respect to the with regard to survivability prediction 142.

According to some embodiments, the cross-modality attention (am) may be represented according to Equation (3):

$${a}_{m} = \frac{{exp W}_{m}^{T} Tanh ( {V}_{m} {f}_{m}^{T} )}{{\sum }_{m = 1}^{M} {exp W}_{m}^{T} Tanh ( {V}_{m} {f}_{m}^{T} )} \qquad Eq . ( 3 )$$

Where m is an integer index, M is an integer representing the number of the input modalities 102/104 (e.g., 2), Wₘ∈R^{L×1} and Vₘ∈R^{L×N} are matrices of trainable parameters in the AMMF circuit 130-1 representing weights of two convolutional layers, L represents the size (e.g., length) of the unimodal embedding output fm (i.e., the size of the first/second intermediate feature vector 112/114), N is the number of output channels of the first layer of the AMMF circuit 130-1 (i.e., the size of the output of the first convolutional layer 302-1), T represents the matrix transpose operation, and Tanh(·) denotes the tangent element-wise non-linear activation function.

The AMMF circuit 130-1 further includes the feature aggregator 312 that applies the attention scores a₁ and a₂ to the corresponding ones of the first and second intermediate feature vectors 112 and 114 and combines the results to generate an aggregate feature vector 132. As operation of the feature aggregator 312 and the resulting aggregate feature vector 132 were described above with respect to FIG. 3A, their description will not be repeated here for sake of brevity.

According to some embodiments, when training the AMMF circuit 130/13-1, a loss function, such as a survival loss function or a Cox loss function, may be utilized to supervise the outcome from the fusion architecture. In the example of survival loss function, the continuous timescale of overall patient survival time in days or months may be partitioned into a plurality of non-overlapping bins (e.g., 4 non-overlapping bins). The negative log-likelihood (NLL) survival loss may be utilized to supervise the training using both censorship status and bin interval labels as a classification task. Survival loss may be flexible for varying sizes of the data with a batch size of 1. In the example of cox loss function, the order of the survival times within a group of samples are supervised using survival time and censor status as the regression task. An observation is said to be censored if the event of interest (e.g., death, relapse, failure) has not occurred or been observed by the end of the study period. Cox loss may utilize a relatively large batch size (e.g., 32, 64, etc.) to achieve better performance. Therefore, the size of the data may be made uniform before being loaded into the model.

In some examples, the first and second modalities may be based on data from patients who received atezolizumab plus carboplatin plus paclitaxel from a phase 3 clinical trial that evaluated the efficacy of adding targeted treatment to programmed cell death ligand 1 (PD-L1) versus the current standard of care in non-small cell lung cancer. A multimodal framework according to some examples may be based on anonymized histopathology images (e.g., from 270 patients) alongside bulk RNA-sequence data.

In some examples, the first modality data (e.g., pathology data) 102 may include tissue image data. For example, H&E-stained whole slide image (WSI) data may be scanned (e.g., at 20× (0.5 micron/pixel)). In some examples, the slides may include manual stroma and tumor region annotations. However, in some embodiments, a pretrained model with U-Net architecture is utilized to classify regions into tumor and stroma. Pixel tiles (e.g., 512×512) may be captured from the annotated/classified WSIs and form the first modality data 102. In some examples, each image tile may be embedded, through the first model 110, by a pretrained model weight with a ResNet-50 backbone into the first intermediate feature vector 112, which may be a 1024-channel feature vector (e.g., a vector of length 1024).

In some examples, the second modality data (e.g., genetics data) 104 includes RNA-sequence data, containing gene expression values along with ensemble gene identifiers. According to some examples, the RNA-sequence data may be preprocessed by (1) mapping all ensemble gene IDs with gene symbols, (2) normalizing RNA-sequence data to generate transcripts-per-million (TPM) expression data, (3) calculating the Z-score for the TPM data, and (4) selecting (e.g., manually selecting) a plurality of genes (e.g., 154 genes) that are most relevant to lung cancer from the curated data.

Such input modality data may be used to test the performance of prediction system 100 with respect to unimodal and fusion systems of the related art.

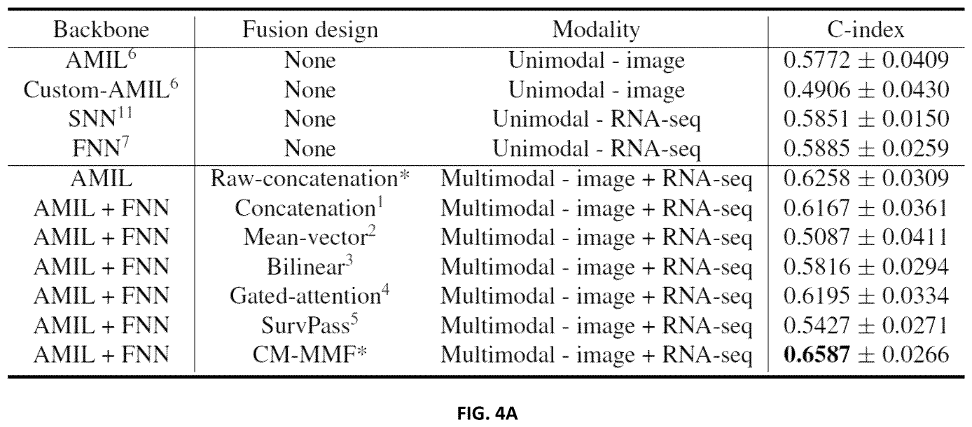

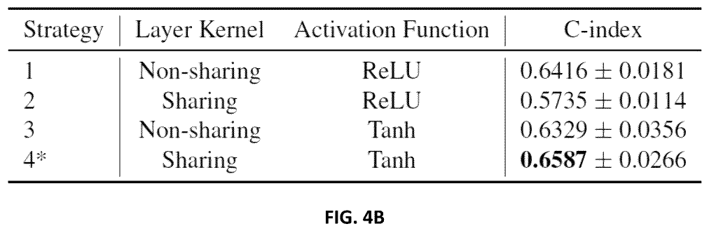

FIG. 4A illustrates a table comparing the performance of prediction system 100 with different systems of the related art across various modalities with different types of loss supervision, according to some embodiments of the present disclosure.

In Table 1, the survival prediction results are evaluated using the concordance index (c-index), which measures the proportion of all possible pairs of observations where the model's predicted values correctly predict the ordering of actual survival times.

As shown in FIG. 4A, most of the fusion designs with multimodal learning achieved superior performance than unimodal learning, demonstrating the capability that infuse the modality-specific knowledge from different modalities. However, the prediction system 100 with cross-modality attention-based multimodal fusion (last row of the table) achieve the highest c-index of about 0.6587 when compared to the other systems of the related art. In the table of FIG. 4A, “raw-concatenation” may refer to a fusion design of the related art in which RNA-seq features are directly concatenated with image features without passing through a feedforward neural network (FNN).

To improve training robustness, Gaussian noise was added to image features and RNA-seq features before being loaded into the AMMF circuit 130-1. All of the models in the table of FIG. 4A were trained over 55 epochs with a learning rate of 0.01 and a batch size of 1 using the ADAM optimizer. Standardization was implemented for the RNA-seq modality, and normalization was deployed to rearrange the feature vectors between 0 and 1 for all modalities before implementing the fusion architecture.

FIG. 4B illustrates a table comparing the performance of prediction system 100 using sharing and non-sharing kernel layers and different activation functions, according to some embodiments of the present disclosure.

In the table of FIG. 4B, various attention mechanism designs with different activation functions (e.g., ReLU and Tanh) were evaluated using the same dataset as FIG. 4A. The attention-based fusion approach according to some embodiments is illustrated in FIG. 4B as being split into two strategies, differentiated by whether or not they shared the kernel weights (see, e.g., the kernel sharing design of AMMF circuit 130-1 and the non-sharing kernel design of the AMMF circuit 130) while learning the embedding features from multiple modalities. As shown by the survival prediction performance in Table 3, in some examples, sharing the kernel weight in the attention-based fusion approach with Tanh activation function (as e.g., shown in FIG. 2) may achieve better performances with a higher mean value of c-index. However, this is merely an example, and the performance of each of the designs in FIG. 4B may change depending on the modality data used.

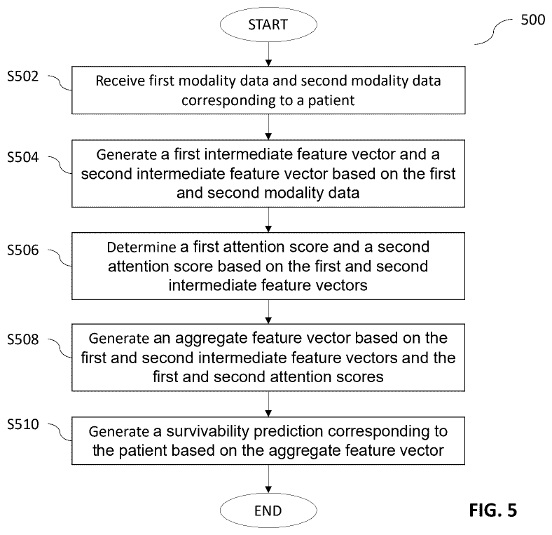

FIG. 5 is a flow diagram illustrating a process 500 of predicting overall survivability of a patient by the prediction system 100, according to some embodiments of the present disclosure.

In some embodiments, the prediction system 100 (e.g., the first and second models 110 and 120) receives first modality data 102 and second modality data 104 corresponding to the patient (S502) and generates a first intermediate feature vector 112 and a second intermediate feature vector 114 based on the first and second modality data 102 and 104 (S504).

The prediction system 100 (e.g., the AMMF circuit 130) then determines a first attention score a₁ and a second attention score a₂ based on the first and second intermediate feature vectors 112 and 114 (S506), and generates an aggregate feature vector 132 based on the first and second intermediate feature vectors 112 and 114 and the first and second attention scores a₁ and a₂ (S508).

In some embodiments, determining the first and second attention scores a₁ and a₂ includes: combining the first and second intermediate feature vectors 112 and 114 to generate a combined feature 116; processing the combined feature 116 by one or more convolution layers 302-1 and 306-1 and an activation layer 304-1 to generate first and second attention values corresponding to the first and second intermediate feature vectors 112 and 114; and converting the first and second attention values to the first and second attention score a₁ and a₂ by a softmax function layer 308-1. In some examples, combining the first and second intermediate feature vectors 112 and 114 includes: stacking the first and second intermediate feature vectors 112 and 114 into a 2-dimensional array that is the combined feature 116. In such examples, the one or more convolution layers 302-1 and 306-1 are configured to perform 2-dimensional convolution on the 2-dimensional array 116.

In some other embodiments, determining the first and second attention scores a₁ and a₂ includes: processing the first intermediate feature vector 112 by one or more first convolution layers 302 and 306 and a first activation layer 304 to generate a first attention value corresponding to the first intermediate feature vector 112; processing the second intermediate feature vector 114 by one or more second convolution layers 302 and 306 and a second activation layer 304 to generate a second attention value corresponding to the second intermediate feature vector 114; and converting the first and second attention values to the first and second attention scores a₁ and a₂ by a softmax function layer 308.

The prediction system 100 (e.g., the classifier 140) then generates a survivability prediction 142 corresponding to the patient based on the aggregate feature vector 132 (S510).

As described above, aspects of some embodiments include an attention-based multi-modal fusion architecture to infuse the knowledge from pathology data and genetic data to achieve improved lung cancer survival predictions. An attention-based multimodal fusion method may achieve superior lung cancer survival prediction with a higher C-index compared to other fusion designs and unimodal learning methods. The attention scores from the fusion layer may demonstrate the importance of each modality for diagnosis, while the instance attention from multiple instance learning (AMIL) can indicate the contribution of each image tile. The cross-modality attention-based multimodal fusion method, according to some embodiments, may outperform other fusion designs and unimodal learning methods. This underscores its capability to integrate modality-specific knowledge from various sources and highlights the functionality of multimodal fusion that takes cross-modality relationships into account.

According to various embodiments of the present disclosure, the prediction system 100 is implemented using one or more processing circuits or electronic circuits configured to perform various operations as described above. Types of electronic circuits may include a central processing unit (CPU), a graphics processing unit (GPU), an artificial intelligence (AI) accelerator (e.g., a vector processor, which may include vector arithmetic logic units configured efficiently perform operations common to neural networks, such dot products and softmax), a field programmable gate array (FPGA), an application specific integrated circuit (ASIC), a digital signal processor (DSP), or the like. For example, in some circumstances, aspects of embodiments of the present disclosure are implemented in program instructions that are stored in a non-volatile computer readable memory where, when executed by the electronic circuit (e.g., a CPU, a GPU, an AI accelerator, or combinations thereof), perform the operations described. The operations performed by the prediction system 200 may be performed by a single electronic circuit (e.g., a single CPU, a single GPU, or the like) or may be allocated between multiple electronic circuits (e.g., multiple GPUs or a CPU in conjunction with a GPU). The multiple electronic circuits may be local to one another (e.g., located on a same die, located within a same package, or located within a same embedded device or computer system) and/or may be remote from one other (e.g., in communication over a network such as a local personal area network such as Bluetooth®, over a local area network such as a local wired and/or wireless network, and/or over wide area network such as the internet, such a case where some operations are performed locally and other operations are performed on a server hosted by a cloud computing service). One or more electronic circuits operating to implement the prediction system 200 may be referred to herein as a computer or a computer system, which may include memory storing instructions that, when executed by the one or more electronic circuits, implement the systems and methods described herein.

The terminology used herein is for the purpose of describing particular embodiments only and is not intended to be limiting of the present invention. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present invention.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present invention refers to “one or more embodiments of the present invention.” As used herein, the terms “use,” “using,” and “used” may be considered synonymous with the terms “utilize,” “utilizing,” and “utilized,” respectively.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Although aspects of some example embodiments of the system and method of quantification of a pathology slide using a cell-based scoring system have been described and illustrated herein, various modifications and variations may be implemented, as would be understood by a person having ordinary skill in the art, without departing from the spirit and scope of embodiments according to the present disclosure. Accordingly, it is to be understood that a pathology slide manufacturing system and method according to the principles of the present disclosure may be embodiment other than as specifically described herein. The disclosure is also defined in the following claims, and equivalents thereof.

What is claimed is:

1. A method of predicting overall survivability of a patient by a prediction system based on machine learning, the method comprising:
receiving, by the prediction system, first modality data and second modality data corresponding to the patient;
generating, by the prediction system, a first intermediate feature vector and a second intermediate feature vector based on the first and second modality data;
determining, by the prediction system, a first attention score and a second attention score based on the first and second intermediate feature vectors;
generating, by the prediction system, an aggregate feature vector based on the first and second intermediate feature vectors and the first and second attention scores; and
generating, by the prediction system, a survivability prediction corresponding to the patient based on the aggregate feature vector.

2. The method of claim 1, wherein the first modality data comprises histology hematoxylin and eosin (H&E) image data, and the second modality data comprises genetic sequencing data.

3. The method of claim 2, wherein the H&E image data comprises a digitized image of a tissue sample of the patient that is stained with hematoxylin and eosin dyes, and
wherein the genetic sequencing data comprises mRNA gene expressions extracted from a tumorous tissue of the patient.

4. The method of claim 1, wherein the receiving the first modality data and the second modality data comprises:
receiving, by a first model of the prediction system, the first modality data; and
receiving, by a second model of the prediction system, the second modality data.

5. The method of claim 4, wherein the generating the first intermediate feature vector and the second intermediate feature vector comprises:
generating, by the first model, the first intermediate feature vector based on the first modality data; and
generating, by the second model, the second intermediate feature vector based on the second modality data.

6. The method of claim 4, wherein the first model comprises an attention-based multiple instance learning (AMIL) model, and the second model comprises a feedforward neural network (FNN) model.

7. The method of claim 1, wherein the determining the first attention score and the second attention score comprises:
combining the first and second intermediate feature vectors to generate a combined feature;
processing the combined feature by one or more convolution layers and an activation layer to generate first and second attention values corresponding to the first and second intermediate feature vectors; and
converting the first and second attention values to the first and second attention score by a softmax function layer.

8. The method of claim 7, wherein the combining the first and second intermediate feature vectors comprises:
stacking the first and second intermediate feature vectors into a 2-dimensional array that is the combined feature, and
wherein the one or more convolution layers are configured to perform 2-dimensional convolution on the 2-dimensional array.

9. The method of claim 7, wherein the activation layer comprises a hyperbolic tangent (Tanh) function layer or a rectified linear unit (ReLU) function layer, and
wherein a summation of the first and second attention scores is equal to 1.

10. The method of claim 7, further comprising:
normalizing values of the first and second intermediate feature vectors before the combining the first and second intermediate feature vectors.

11. The method of claim 1, wherein the determining the first attention score and the second attention score comprises:
processing the first intermediate feature vector by one or more first convolution layers and a first activation layer to generate a first attention value corresponding to the first intermediate feature vector;
processing the second intermediate feature vector by one or more second convolution layers and a second activation layer to generate a second attention value corresponding to the second intermediate feature vector; and
converting the first and second attention values to the first and second attention scores by a softmax function layer.

12. The method of claim 11, wherein each of the first and second activation layers comprises a hyperbolic tangent (Tanh) function layer or a rectified linear unit (ReLU) function layer, and
wherein a summation of the first and second attention scores is equal to 1.

13. The method of claim 11, wherein the one or more first convolution layers comprise kernel values that are not the same as those of the one or more second convolution layers.

14. The method of claim 1, wherein the generating the aggregate feature vector comprises:
scaling the first intermediate feature vector based on the first attention score to generate a first scaled intermediate feature vector;
scaling the second intermediate feature vector based on the second attention score to generate a second scaled intermediate feature vector; and
aggregating the first and second scaled intermediate feature vectors to generate the aggregate feature vector.

15. The method of claim 14, wherein the aggregating the first and second scaled intermediate feature vectors comprises:
concatenating the first and second scaled intermediate feature vectors to generate the aggregate feature vector, the aggregate feature vector having a length equal to a sum of lengths of the first and second scaled intermediate feature vectors.

16. The method of claim 1, wherein the generating the survivability prediction comprises:
generating, by a classifier of the prediction system, the survivability prediction corresponding to the patient based on the aggregate feature vector,
wherein the survivability prediction corresponds to an overall survivability of the patient.

17. The method of claim 16, wherein the survivability prediction comprises a range of values from a plurality of sequential ranges of values.

18. The method of claim 1, further comprising:
transmitting the survivability prediction to a display device for display to a user.

19. A prediction system for predicting overall survivability of a patient based on machine learning, the prediction system comprising:
a first model configured to receive first modality data corresponding to the patient and to generate a first intermediate feature vector based on the first modality data;
a second model configured to receive second modality data corresponding to the patient and to generate a second intermediate feature vector based on the second modality data; and
an attention-based multimodal fusion circuit configured to determine a first attention score and a second attention score based on the first and second intermediate feature vectors, and to generate an aggregate feature vector based on the first and second intermediate feature vectors and the first and second attention scores.

20. The prediction system of claim 19, further comprising:
a classifier configured to receive the aggregate feature vector and to generate a survivability prediction corresponding to the patient based on the aggregate feature vector.
